(19) United States
(12) Patent Application Publication
(10) Pub. No.: US 2026/0115082 A1
(43) Pub. Date: Apr. 30, 2026

(54) DEVICE AND METHOD FOR CONTROLLING UPPER LIMB REHABILITATION EXOSKELETON AND COMPUTER-READABLE STORAGE

(71) Applicant: Futronics (NA) Corporation, Pasadena, CA (US)

(72) Inventors: WENDA XU, Pasadena, CA (US); Yunfei Guo, Pasadena, CA (US); Fulan Li, Pasadena, CA (US); Fungyun Zhao, Pasadena, CA (US); Chengkun Zhang, Pasadena, CA (US)

(21) Appl. No.: 18/929,529

(22) Filed: Oct. 28, 2024

(51) Int. Cl. A61H 1/02; G16H 20/30; G16H 40/67

(52) U.S. Cl. CPC A61H 1/0274; G16H 20/30; G16H 40/67; A61H 2201/1207; A61H 2201/1676; A61H 2201/5007; A61H 2201/5061; A61H 2201/5064

(57) ABSTRACT

A control device for an upper limb rehabilitation exoskeleton is provided. The exoskeleton includes a number of joints, each of which includes an encoder and a torque sensor. The control device includes a processor electrically coupled to the encoders and the torque sensors of the joints of the exoskeleton. For each joint of the joints, the processor is to: receive a real-time position of the joint from the encoder of the joint and a real-time torque of the joint from the torque sensor of the joint, compare the real-time torque of the joint with a desired torque of the joint at the real-time position, and control an output torque of the joint according to a comparison result, such that the output torque of the joint at the real-time position is equal to the desired torque.

### TECHNICAL FIELD

The present disclosure generally relates to robotic exoskeletons, and in particular relates to a device and method for controlling an upper limb rehabilitation exoskeleton and computer-readable storage medium.

### BACKGROUND

Robotic exoskeletons have started to become a recent trend in rehabilitation, specifically in upper and lower extremity rehabilitation. The development of exoskeletons requires a delicate balance of weight, power, human experience, motion and others. Exoskeletons are employed in physical therapy rehabilitation to help improve muscle control and prevent muscle atrophy in disabled patients.

Transparent mode represents a pivotal feature in the realm of upper limb rehabilitation exoskeletons. This mode offers dual functionalities: Coriolis and gravity compensation, and assist-as-needed force assistance. Gravity compensation entails offsetting the weight of both the rehabilitation exoskeleton and the user's limbs. With this feature, when the user applies no force to the rehabilitation exoskeleton, it remains in its current position.

Assist-as-needed functionality enables effortless operation of the rehabilitation exoskeleton. It can discern the user's intentions and deliver assistance or resistance forces in the desired direction of movement. When the user's intended force aligns with the expected acceleration, the exoskeleton achieves transparency from the user's perspective. If the current acceleration exceeds expectations, the desired force assistance will be provided. Conversely, if the acceleration is lower than expected, force resistance will be enacted.

Many researchers have tackled transparent control of exoskeletons with precise dynamic models. However, real-world exoskeletons often encounter difficulties in measuring friction forces due to manufacturing and assembly issues. Additionally, components may suffer from backlash or minor deformations, deviating from the expected dynamic model. Researchers typically address these mismatches by either extensively calibrating the dynamic model or incorporating additional sensors. However, the inclusion of extra sensors escalates costs and introduces further complexity.

Therefore, there is a need to provide a device and method for controlling an upper limb rehabilitation exoskeleton to overcome the above-mentioned problems.

### BRIEF DESCRIPTION OF DRAWINGS

Many aspects of the present embodiments can be better understood with reference to the following drawings. The components in the drawings are not necessarily drawn to scale, the emphasis instead being placed upon clearly illustrating the principles of the present embodiments. Moreover, in the drawings, all the views are schematic, and like reference numerals designate corresponding parts throughout the several views.

FIG. 1 is a schematic isometric view of an upper limb rehabilitation exoskeleton according to one embodiment.

FIG. 2 is another schematic isometric view of the upper limb rehabilitation exoskeleton viewed from a different perspective.

FIG. 3 is a schematic diagram of the application scenario of the exoskeleton shown in FIGS. 1 and 2.

FIG. 4 is a schematic block diagram of a control device for an upper limb rehabilitation exoskeleton according to one embodiment.

FIG. 5 is a flowchart of a method for controlling an upper limb rehabilitation exoskeleton according to one embodiment.

FIG. 6 is another flowchart of a method for controlling an upper limb rehabilitation exoskeleton according to one embodiment.

FIG. 7 is a flowchart of a method for adjusting output torques of the joints of an upper limb rehabilitation exoskeleton according to one embodiment.

FIG. 8 is a schematic diagram showing the control process for an upper limb rehabilitation exoskeleton according to one embodiment.

FIG. 9 is another schematic block diagram of the control device for an upper limb rehabilitation exoskeleton according to one embodiment.

### DETAILED DESCRIPTION

The disclosure is illustrated by way of example and not by way of limitation in the figures of the accompanying drawings, in which like reference numerals indicate similar elements. It should be noted that references to “an” or “one” embodiment in this disclosure are not necessarily to the same embodiment, and such references can mean “at least one” embodiment.

Although the features and elements of the present disclosure are described as embodiments in particular combinations, each feature or element can be used alone or in other various combinations within the principles of the present disclosure to the full extent indicated by the broad general meaning of the terms in which the appended claims are expressed.

The following dynamic equation, which is a mathematical model that describes the physical motion of the entire exoskeleton, depicts the force acting on joints of the exoskeleton combined with user's arms: I(qᵢ)\ddot{q}ᵢ+C(qᵢ,\dot{q}ᵢ)+G(qᵢ)=uᵢ, where G represents the gravity force exerted on the joint, the force applied to the joint is a function of the angle and includes the vector of gravity; C represents Coriolis force that is a function of angular velocity and includes the Coriolis vector; I represents the moment of inertia at given configuration; u represents output torque on each joint, q represents a joint angle that is the angle between two mechanical structures connected by a motor joint, which changes as the motor rotates, and i∈{exoskeleton, user}. When i=4, it means that there are four joints and four motors. When the user's intended force aligns with the expected acceleration \ddot{q}, the exoskeleton achieves transparency from the user's perspective. If the current acceleration \ddot{q} exceeds expectations, the desired force assistance will be provided. Conversely, if the acceleration is lower than expected, force resistance will be enacted.

In one embodiment, the present disclosure provides a control device for an upper limb rehabilitation exoskeleton (hereinafter referred to as “the exoskeleton”) for controlling the output torque of each joint of the exoskeleton to compensate for the user's intended force, enabling a user to easily rotate the joints of the exoskeleton during rehabilitation of an upper limb of the user.

Assist-as-needed functionality enables effortless operation of the rehabilitation exoskeleton. It can discern the user's intentions and deliver assistance or resistance forces in the desired direction of movement.

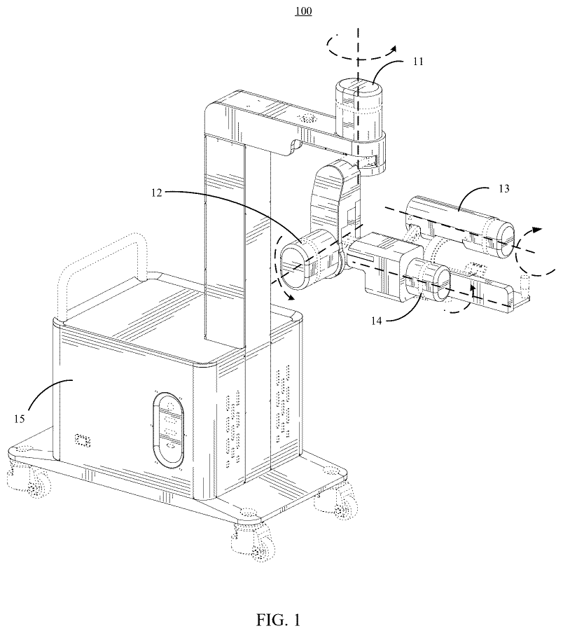

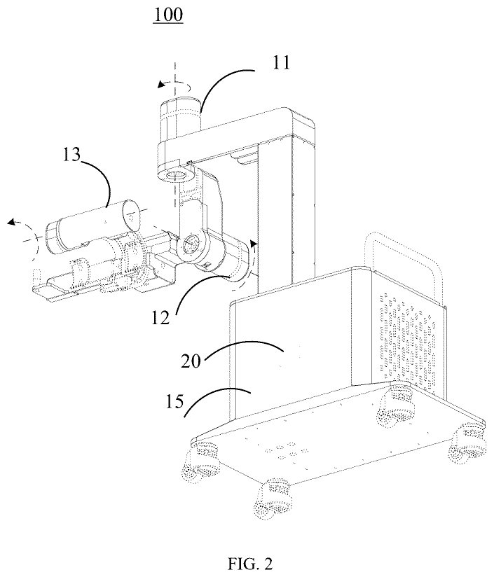

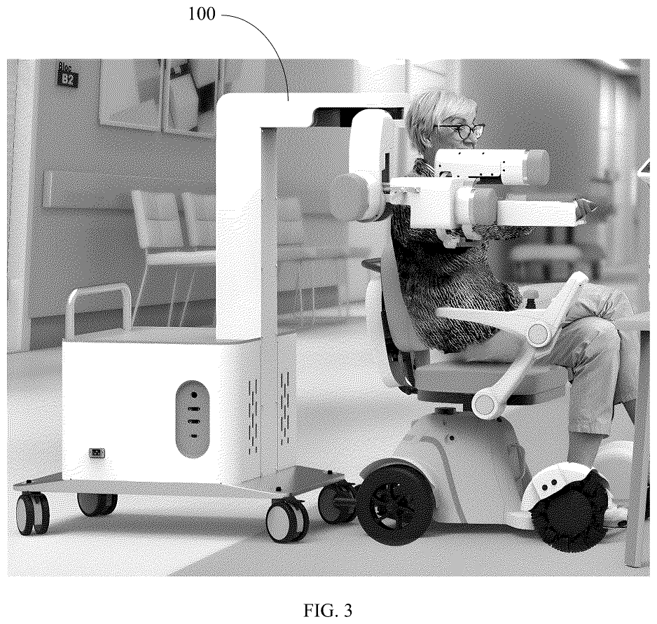

FIGS. 1 and 2 are schematic isomeric views of the exoskeleton according to one embodiment. The exoskeleton 100 may have 4 degrees of freedom (DoF), including a first joint 11, a second joint 12, a third joint 13 and a fourth joint 14. The directions of rotation of the four joints are indicated by the dotted arrows in FIGS. 1 and 2, and the axis of rotation of each joint is indicated by the straight dotted lines in FIGS. 1 and 2. The four joints can be driven in reverse. FIG. 3 is a schematic diagram of an application scenario in which a user uses the exoskeleton for rehabilitation under the control of the control device for the exoskeleton. The exoskeleton has an arm-like mechanism that includes the four joints and can be attached around and to an arm of a user and can move together with the arm when the user applies a force to the mechanism of the exoskeleton. Under the force applied by the user, the exoskeleton 100 can assist the user in exercising the upper limb (i.e., the arm) through the rotation of the four joints. It should be noted that the control method described in the embodiments of the present disclosure may be applicable to exoskeletons using joints that cannot be backdriven.

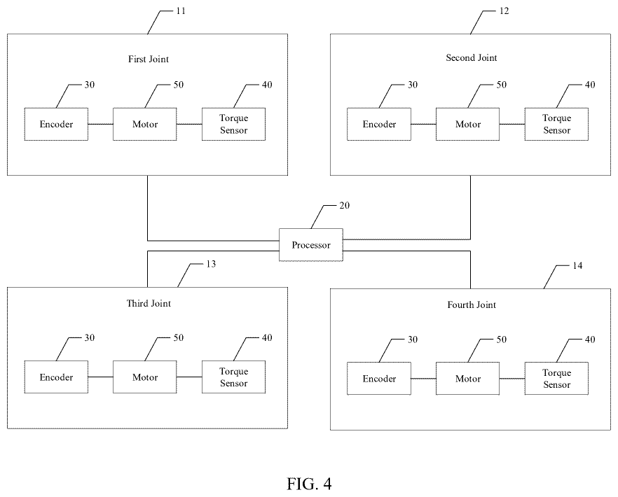

Referring to FIG. 4, in one embodiment, the control device may include a processor 20 that is electrically coupled to an encoder 30 and a torque sensor 40 that are arranged in each joint of the exoskeleton. Each joint of the exoskeleton may further include a motor 50 that is electrically coupled to the encoder 30 and the torque sensor 40 of the joint and the processor 50. In one embodiment, the processor 20 may be arranged in a base 15 of the exoskeleton 100.

In the embodiment as shown in FIG. 2, each of the first joint 11 the second joint 12, the third joint 13 and the fourth joint 14 may include the encoder 30 and the torque sensor 40 and the motor 50 that is electrically coupled to the encoder 30 and the torque sensor 40. When the user uses the exoskeleton for rehabilitation exercises, the encoder 30 in each joint measures the real-time position of the joint including the encoder, and the real-time position of the joint can be obtained by measuring the position of the shaft of the motor in the joint. The torque sensor 40 in each joint is to measure the real-time torque of the joint including the torque sensor 40, and the real-time torque of the joint can be obtained by measuring the real-time output torque of the motor in the joint. In one embodiment, the torque sensors can be one-axis torque sensors.

The processor 20 is to calculate a desired torque of each joint of the exoskeleton 100. The desired torque is based on gravity compensation and the desired torque for the user's motion. When the user uses the exoskeleton, the user applies appropriate force to the exoskeleton, more specifically to the arm-like mechanism of the exoskeleton, so that the joints of the exoskeleton reach the desired positions with the desired torques, thereby achieving the user's upper limb rehabilitation training purpose.

For each joint of the joints, the processor 20 is to calculate the desired torque Tau_pred of the joint according to a joint position P, a joint velocity V and a joint acceleration A of the joint when an upper limb of the user is fixed on the exoskeleton and the user applies no force to the exoskeleton.

The joint position P can be pre-set, and when the set joint position is reached, the corresponding joint velocity V and joint acceleration A are obtained to adjust the joint torque.

There are multiple joint positions P, and each corresponds to its own joint velocity V and joint acceleration A. In the process of controlling the exoskeleton, if the joint position P1 is detected, the torque is adjusted according to the difference between the detected joint torque T1 and the corresponding desired torque Tp1. If the joint position P2 is detected, the torque is adjusted according to the difference between the detected joint torque T2 and the corresponding desired torque Tp2, and a similar process will be performed for other joint positions. That is, controlling the joint output torque of the exoskeleton is a dynamic process.

In another embodiment, for each joint of the joints, the processor 20 is to calculate the sum of the torque C(v) of the Coriolis force generated by the joint velocity, the torque Tau_(a) of the force generated by the joint acceleration, and the torque pred(p) of the joint for holding the upper limb of the user and the exoskeleton in place at the joint position as the desired torque Tau_pred of the joint. The equation for calculating the sum of these torques are as follows: Tau_pred=pred(p)+C(v)+Tau(a), where pred(p) is the torque required to hold a joint of the exoskeleton to which the upper limb of the human body is attached at position p without considering motion, and can be directly calculated by using precise dynamic models, for example, in 3D CAD design software SolidWorks.

The equation above for the desired torque does not take into account factors such as friction and is a rough calculation model. As a prediction model for the desired torque, the rough calculation model can be used in combination with user applications to continuously improve the accuracy of the calculation model.

In another embodiment, the processor 20 is to: obtain the real-time position of each joint measured by the encoder located in each joint, and the real-time torque Tau_measured of each joint measured by the torque sensor 30 at the real-time position, obtain the desired torque Tau_pred of each joint at the real-time position, and compare the real-time torque Tau_measured and the desired torque Tau_pred of each joint. If the real-time torque of the joint at the current measured real-time position is less than or greater than the desired torque of the joint at the real-time position, the difference (Tau_pred−Tau_measured) between the real-time torque and the desired torque of the joint will be calculated, and a control instruction will be sent to the joint according to the difference to adjust the output torque of the joint so that the output torque of the joint at the measured position is equal to the desired torque. If the real-time torque of the joint is equal to the desired torque of the joint, the output torque of the joint will remain unchanged.

In one embodiment, for each joint, the processor 20 is to map the difference between the real-time torque and the desired torque of the joint to the output parameters for the motor of the joint through a transfer function TF, and output control instructions containing the output parameters for the motor to the motor of the joint. The output parameters of the motor include control signals sent to the motor, such as current, speed or position.

The transfer function TF is to map the torque difference to the motor control output. Different torque differences can be used to generate functions that control the output parameters for the motor. The resistance or assistance level to the joint can be adjusted by adjusting this transfer function.

In one embodiment, the equation for adjusting the output torque of the joint is as follows: Motor_output=(Tau_pred−Tau_measured)*TF.

Specifically, if the real-time torque of a joint measured at a real-time position is less than the desired torque at the real-time position, the user's upper limb strength is insufficient to make the joint rotate at the acceleration corresponding to the desired torque. In this case, the processor 20 will send a control instruction to the motor of the joint, and increases the output torque of the motor according to the difference between the real-time torque and the desired torque, so that the torque of the joint is equal to the desired torque. This is equivalent to providing assistance for the force applied by the user, so that the user can rotate the joint to the real-time position of the joint with the acceleration corresponding to the desired torque even if the user uses insufficient upper limb strength, thereby realizing transparent control of the exoskeleton on the user side.

On the other hand, if the real-time torque of a joint measured at a real-time position is greater than the desired torque, the user's upper limb strength can make the joint rotate at an acceleration greater than the desired torque. In this case, the processor 20 will send a control instruction to the motor of the joint, and reduces the output torque of the motor according to the difference between the real-time torque and the desired torque, so that the torque of the joint is equal to the desired torque. This is equivalent to adding resistance to the force applied by the user, so that even if the user's upper limb strength is too large, the joint can be rotated to the real-time position of the joint with an acceleration corresponding to the desired torque, thereby realizing transparent control of the exoskeleton on the user side.

For example, when an upper limb of user A is fixed on the exoskeleton and user A does not apply pressure to the exoskeleton, in order to realize transparent control of the exoskeleton by user A without considering the motion of user A and the total weight of the upper limb of user A and the exoskeleton, the four joints of the exoskeleton each are controlled to rotate to multiple desired positions at a certain speed and a certain acceleration to generate desired torques. For example, the first joint can rotate to position p11 at v11 and a11, generating a desired torque Tau_pr11, and can rotate to position p12 at v12 and a12, generating a desired torque Tau_pr12. Similarly, the second joint can rotate to position p21 at v21 and a21, generating a desired torque Tau_pr21, and can rotate to position p22 at v22 and a22, generating a desired torque Tau_pr22. Similarly, the third and fourth joints can generate their own desired torques. The processor can then calculate the desired torque for each joint at multiple positions using the equation Tau_pred=pred(p)+C(v)+Tau(a).

When user A attempts to perform rehabilitation training on an upper limb, the upper limb will be attached to the arm-like mechanism of the exoskeleton so that the upper limb can apply force to make the arm-like mechanism of the exoskeleton move together with the upper limb of user A. The encoder in each joint measures the current real-time position of the joint, and the torque sensor in each joint measures the real-time torque of the joint. The processor obtains the desired torques corresponding to the real-time positions based on the desired torque of different positions calculated previously, and compares the desired torques with the real-time torques. For each joint, if the real-time torque is less than the desired torque, the output torque of the current joint will be increased according to the difference between the two torques so that the output torque will be equal to the desired torque, providing assistance for the force applied by the user. If the real-time torque is greater than the desired torque, the output torque of the current joint will be reduced according to the difference between the two torques so that the output torque will be equal to the desired torque, providing resistance for the force applied by the user.

In summary, the control device for the exoskeleton discussed above can track the real-time positions of the joints by the encoder in each joint of the exoskeleton and measuring the real-time output torque of the joint by the torque sensor in each joint of the exoskeleton. For each joint of the exoskeleton, the real-time output torque of the joint will be compared with the desired output torque of the joint at the real-time position. The desired output torques are obtained on the basis of taking into account the gravity of the user's upper limb and the exoskeleton and the user's motion. The output torques of the joints of the exoskeleton can be adjusted according to the difference between the real-time output torques and the desired output torques, which can achieve transparent control of the exoskeleton on the user side. In addition, the use of encoders and torque sensors reduces the manufacturing cost of the control device and reduces the complexity of the control device.

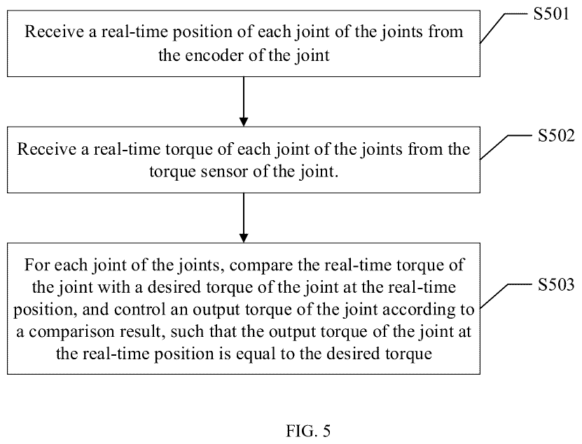

In one embodiment, a method for controlling the exoskeleton is provided. The method can be implemented by the control device discussed in the foregoing embodiments. Referring to FIG. 5, in one embodiment, the method may include steps S501 to S503.

Step S501: Receive a real-time position of each joint of the joints from the encoder of the joint.

For each joint of the exoskeleton, the processor of the control device may receive the real-time position of the joint from the encoder of the joint. The encoder measures the position of the shaft of the motor in the joint and the position of the shaft of the motor in the joint is determined as the real-time position of the joint.

Step S502: Receive a real-time torque of each joint of the joints from the torque sensor of the joint.

For each joint of the exoskeleton, the processor of the control device may receive a real-time torque of the joint from the torque sensor of the joint. The torque sensor measures the real-time output torque of the motor in the joint and the real-time output torque of the motor in the join is determined as the real-time torque. In one embodiment, the torque sensor may be a one-axis torque sensor.

Step S503: For each joint of the joints, compare the real-time torque of the joint with a desired torque of the joint at the real-time position, and control an output torque of the joint according to a comparison result, such that the output torque of the joint at the real-time position is equal to the desired torque.

For each joint of the exoskeleton, the processor of the control device may compare the real-time torque of the joint with a desired torque of the joint at the real-time position, and control the output torque of the joint according to a comparison result.

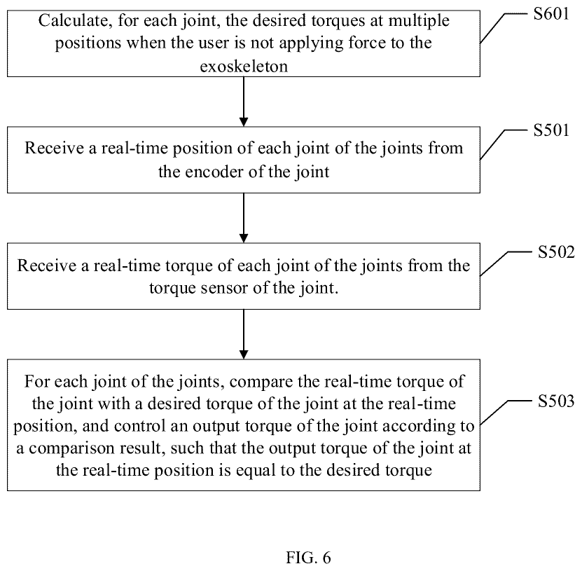

Referring to FIG. 6, in one embodiment, the method may further include step S601 before step S501.

Step S601: Calculate, for each joint, the desired torques at multiple positions when the user is not applying force to the exoskeleton.

Specifically, the desired torque of the joint is calculated according to a joint position P, a joint velocity V and a joint acceleration A of the joint when an upper limb of the user is fixed on the exoskeleton and the user applies no force to the exoskeleton. The sum of the torque of the Coriolis force generated by the joint velocity, the torque of the force generated by the joint acceleration, and the torque of the joint for holding the upper limb of the user and the exoskeleton in place at the joint position is calculated and determined as the desired torque. The calculated desired torque will be stored in the processor or in a storage medium electrically coupled to the processor for the processor to access.

In one embodiment, step S503 specifically involves: for each joint of the joints, send a control instruction to the joint according to a difference between the real-time torque and the desired torque of the joint to adjust the output torque of the joint, in response to the real-time torque of the joint at the real-time position being less than or greater than the desired torque of the joint at the real-time position.

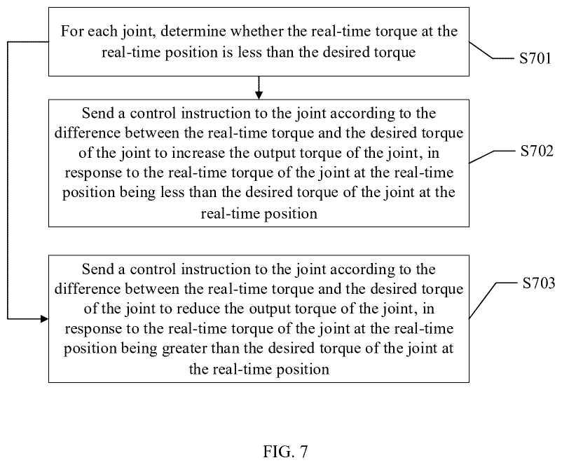

Referring to FIG. 7, in one embodiment, step S503 may further include the following steps S701 to S703.

Step S701: For each joint, determine whether the real-time torque at the real-time position is less than the desired torque.

For each joint, the processor of the control device determines whether the real-time torque of the joint at the measured real-time position is less than the desired torque of the joint at the real-time position.

Step S702: Send a control instruction to the joint according to the difference between the real-time torque and the desired torque of the joint to increase the output torque of the joint, in response to the real-time torque of the joint at the real-time position being less than the desired torque of the joint at the real-time position.

Specifically, if the real-time torque of the joint measured at the real-time position is less than the desired torque of the joint at the real-time position, a control instruction will be sent to the motor of the joint to increase the output torque of the motor according to the difference between the real-time torque and the desired torque, thereby increasing the real-time torque of the joint.

Step S703: Send a control instruction to the joint according to the difference between the real-time torque and the desired torque of the joint to reduce the output torque of the joint, in response to the real-time torque of the joint at the real-time position being greater than the desired torque of the joint at the real-time position.

Specifically, if the real-time torque of the joint measured at the real-time position is greater than the desired torque of the joint at the real-time position, a control instruction will be sent to the motor of the joint to reduce the output torque of the motor according to the difference between the real-time torque and the desired torque, thereby reducing the real-time torque of the joint.

In one embodiment, the difference between the real-time torque and the desired torque of each joint of the joints is mapped into output parameters for a motor of the joint through a transfer function, and control instructions are output to the motor of each joint of the joints, respectively. In one embodiment, the control instructions may include the output parameters of the motors. The output parameters of the motor may include control signals sent to the motor, such as current, speed or position.

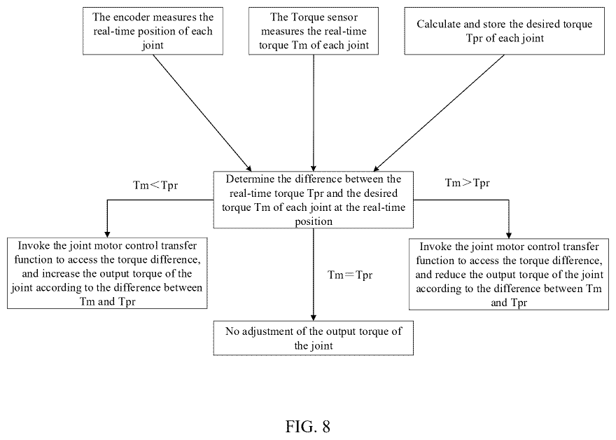

FIG. 8 is an exemplary flowchart of the method for controlling the exoskeleton according to one embodiment. The processor calculates and stores the desired torques of each joint at multiple positions. The processor then obtains the real-time position of each joint measured by the encoder in the joint and obtains the real-time torque of the joint measured by the torque sensor in the joint, and compares the real-time torque and the desired torque of each joint at the real-time position. If the real-time torque is less than the desired torque, the torque difference will be invoked to the joint motor control transfer function TF, and the difference between the real-time torque and the desired torque will be mapped to the output of the motor using the transfer function TF to increase the output torque of the joint. If the real-time torque is greater than the desired torque, the transfer function TF will be invoked. The transfer function TF is then used to map the difference between the real-time torque and the desired torque to the motor output, thereby reducing the output torque of the joint. If the real-time torque equals the desired torque, the output torque of the joint will not be adjusted.

The method described in the foregoing embodiment features a versatile control pipeline. This pipeline captures desired torque data from the 1-axis torque sensor of each joint of the exoskeleton in configuration space. It then compares the desired torque with the measured real-time torque and adjusts the motor output of the joint accordingly to achieve transparency control. Each component model within this control pipeline is adjustable to suit different configurations. The core of the control process includes: data-driven joint torque estimator and transfer function for torque difference to motor control. The joint torque estimator is to combine a rough dynamic model with the measured torque data to predict the desired torque of the exoskeleton. The transfer function is to map the torque difference to the motor control output. This function uses different torques to produce the control output. The resistance or assistance level can be adjusted by adjusting this transfer function. Additionally, a data-driven approach is proposed to estimate joint torque. Paired with a less precise dynamic model, this approach aims to yield outcomes akin to those achieved with an accurate dynamic model, offering a viable alternative.

The application of the foregoing control process can help promote the advancement of upper limb rehabilitation exoskeletons and be extended to other similar rehabilitation robots, which can also bring higher value to healthcare applications.

According to the method for controlling the exoskeleton in the foregoing embodiments, an encoder can be arranged in each joint of the exoskeleton to track the real-time position of the joint and a torque sensor can be employed to measure the real-time output torque of the joint. For each joint of the exoskeleton, the real-time joint output torque of the exoskeleton and the desired output torque of the joint at the real-time position can be compared. The desired output torques are obtained on the basis of taking into account the gravity of the user's upper limb and the exoskeleton and the user's motion. The output torque of the joint of the exoskeleton is controlled according to their difference, which reduces the complexity of transparent control of the exoskeleton on the user side and improves the control efficiency.

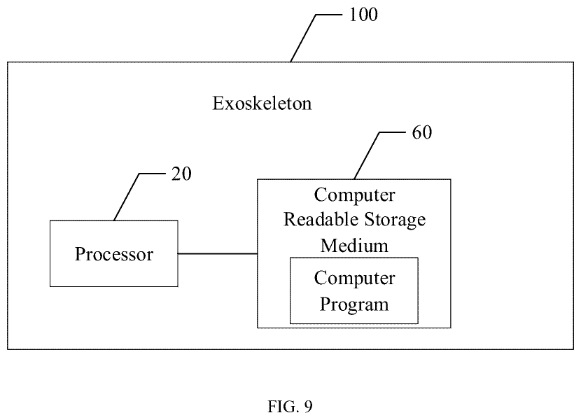

Another aspect of the present disclosure is directed to a non-transitory computer-readable medium. Referring to FIG. 9, in one embodiment, the non-transitory computer readable storage medium 60 is disposed in the exoskeleton 100 and is electrically coupled to the processor 20. The non-transitory computer storage medium 60 stores a computer program, which, when executed by the processor 20, causes the processor 20 to perform the method for controlling the exoskeleton as discussed in the foregoing embodiments.

The computer-readable medium may include volatile or non-volatile, magnetic, semiconductor, tape, optical, removable, non-removable, or other types of computer-readable medium or computer-readable storage devices. For example, the computer-readable medium may be the storage device or the memory module having the computer instructions stored thereon, as disclosed. In some embodiments, the computer-readable medium may be a disc or a flash drive having the computer instructions stored thereon.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the invention and its practical applications, to thereby enable others skilled in the art to best utilize the invention and various embodiments with various modifications as are suited to the particular use contemplated.

What is claimed is:

1. A control device for an upper limb rehabilitation exoskeleton that comprises a plurality of joints, each joint of the joints comprising an encoder and a torque sensor, the control device comprising:
a processor electrically coupled to the encoders and the torque sensors of the joints of the upper limb rehabilitation exoskeleton;
wherein, for each joint of the joints, the processor is configured to: receive a real-time position of the joint from the encoder of the joint and a real-time torque of the joint from the torque sensor of the joint, compare the real-time torque of the joint with a desired torque of the joint at the real-time position, and control an output torque of the joint according to a comparison result, such that the output torque of the joint at the real-time position is equal to the desired torque.

2. The control device of claim 1, wherein, for each joint of the joints, the processor is configured to: calculate the desired torque of the joint according to a joint position, a joint velocity and a joint acceleration of the joint when an upper limb of a user is fixed on the upper limb rehabilitation exoskeleton and the user applies no force to the upper limb rehabilitation exoskeleton.

3. The control device of claim 1, wherein the desired torque of each joint of the joints is calculated according to the following equation: Tau_pred=pred(p)+C(v)+Tau(a), where pred(p) represents torques of the joints for holding the upper limb of the user and the upper limb rehabilitation exoskeleton in place at the joint positions, C(v) represents torques of Coriolis forces generated by the joint velocities, and Tau(a) represents torques of forces generated by the joint accelerations.

4. The control device of claim 1, wherein for each joint of the joints, the processor is configured to send a control instruction to the joint according to a difference between the real-time torque and the desired torque of the joint to adjust the output torque of the joint to be equal to the desired torque, in response to the real-time torque of the joint at the real-time position being less than or greater than the desired torque of the joint at the real-time position.

5. The control device of claim 4, wherein the processor is configured to send a control instruction to the joint according to the difference between the real-time torque and the desired torque of the joint to increase the output torque of the joint, in response to the real-time torque of the joint at the real-time position being less than the desired torque of the joint at the real-time position; and the processor is configured to send a control instruction to the joint according to the difference between the real-time torque and the desired torque of the joint to reduce the output torque of the joint, in response to the real-time torque of the joint at the real-time position being greater than the desired torque of the joint at the real-time position.

6. The control device of claim 2, wherein the processor is further configured to map a difference between the real-time torque and the desired torque of each joint of the joints into output parameters for a motor of the joint through a transfer function, and output control instructions to the motor of each joint of the joints, respectively, and the control instructions comprise the output parameters for the motors.

7. The control device of claim 6, wherein the output parameters comprise speed, position or current sent to the motor of each joint of the joints.

8. The control device of claim 1, wherein the torque sensors are one-axis torque sensors.

9. A computer-implemented method for controlling an upper limb rehabilitation exoskeleton that comprises a plurality of joints, each joint of the joints comprising an encoder and a torque sensor, the method comprising:
receiving a real-time position of each joint of the joints from the encoder of the joint;
receiving a real-time torque of each joint of the joints from the torque sensor of the joint; and
for each joint of the joints, comparing the real-time torque of the joint with a desired torque of the joint at the real-time position, and controlling an output torque of the joint according to a comparison result, such that the output torque of the joint at the real-time position is equal to the desired torque.

10. The method of claim 9, further comprising, before receiving the real-time position of each joint of the joints from the encoder of the joint, calculating the desired torque of the joint according to a joint position, a joint velocity and a joint acceleration of the joint when an upper limb of a user is fixed on the upper limb rehabilitation exoskeleton and the user applies no force to the upper limb rehabilitation exoskeleton.

11. The method of claim 10, wherein the desired torque of each joint of the joints is calculated according to the following equation: Tau_pred=pred(p)+C(v)+Tau(a), where pred(p) represents torques of the joints for holding the upper limb of the user and the upper limb rehabilitation exoskeleton in place at the joint positions, C(v) represents torques of Coriolis forces generated by the joint velocities, and Tau(a) represents torques of forces generated by the joint accelerations.

12. The method of claim 9, wherein comparing the real-time torque of the joint with the desired torque of the joint at the real-time position, and controlling the output torque of the joint according to the comparison result comprise: for each joint of the joints, sending a control instruction to the joint according to a difference between the real-time torque and the desired torque of the joint to adjust the output torque of the joint to be equal to the desired torque, in response to the real-time torque of the joint at the real-time position being less than or greater than the desired torque of the joint at the real-time position.

13. The method of claim 12, wherein sending the control instruction to the joint according to the difference between the real-time torque and the desired torque of the joint to adjust the output torque of the joint to be equal to the desired torque, in response to the real-time torque of the joint at the real-time position being less than or greater than the desired torque of the joint at the real-time position, comprises:
sending a control instruction to the joint according to the difference between the real-time torque and the desired torque of the joint to increase the output torque of the joint, in response to the real-time torque of the joint at the real-time position being less than the desired torque of the joint at the real-time position; and
sending a control instruction to the joint according to the difference between the real-time torque and the desired torque of the joint to reduce the output torque of the joint, in response to the real-time torque of the joint at the real-time position being greater than the desired torque of the joint at the real-time position.

14. The method of claim 9, wherein controlling the output torque of the joint according to the comparison result comprises: mapping a difference between the real-time torque and the desired torque of each joint of the joints into output parameters for a motor of the joint through a transfer function, and outputting control instructions to the motor of each joint of the joints, respectively; and wherein the control instructions comprise the output parameters for the motors.

15. The method of claim 14, wherein the output parameters comprise speed, position or current sent to the motor of each joint of the joints.

16. The method of claim 9, wherein the torque sensors are one-axis torque sensors.

17. A non-transitory computer-readable storage medium storing instructions that, when executed by at least one processor of a control device for an upper limb rehabilitation exoskeleton that comprises a plurality of joints each comprising an encoder and a torque sensor, cause the at least one processor to perform a method for controlling the upper limb rehabilitation exoskeleton, the method comprising:
receiving a real-time position of each joint of the joints from the encoder of the joint;
receiving a real-time torque of each joint of the joints from the torque sensor of the joint; and
for each joint of the joints, comparing the real-time torque of the joint with a desired torque of the joint at the real-time position, and controlling an output torque of the joint according to a comparison result, such that the output torque of the joint at the real-time position is equal to the desired torque.

18. The non-transitory computer-readable storage medium of claim 17, wherein the method further comprises, before receiving the real-time position of each joint of the joints from the encoder of the joint, calculating the desired torque of the joint according to a joint position, a joint velocity and a joint acceleration of the joint when an upper limb of a user is fixed on the upper limb rehabilitation exoskeleton and the user applies no force to the upper limb rehabilitation exoskeleton.

19. The non-transitory computer-readable storage medium of claim 18, wherein the desired torque of each joint of the joints is calculated according to the following equation: Tau_pred=pred(p)+C(v)+Tau(a), where pred(p) represents torques of the joints for holding the upper limb of the user and the upper limb rehabilitation exoskeleton in place at the joint positions, C(v) represents torques of Coriolis forces generated by the joint velocities, and Tau(a) represents torques of forces generated by the joint accelerations.

20. The non-transitory computer-readable storage medium of claim 19, wherein comparing the real-time torque of the joint with the desired torque of the joint at the real-time position, and controlling the output torque of the joint according to the comparison result comprise: for each joint of the joints, sending a control instruction to the joint according to a difference between the real-time torque and the desired torque of the joint to adjust the output torque of the joint to be equal to the desired torque, in response to the real-time torque of the joint at the real-time position being less than or greater than the desired torque of the joint at the real-time position.
